(19) United States
(12) Patent Application Publication
(10) Pub. No.: US 2009/0169572 A1
(43) Pub. Date: Jul. 2, 2009

(54) METHODS FOR DAMAGING CELLS USING EFFECTOR FUNCTIONS OF ANTI-CDH3 ANTIBODIES

(75) Inventors: Shuichi Nakatsuru, Kanagawa (JP); Megumi Yoshikawa, Kanagawa (JP); Shinichi Hiroshima, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc,, Kawasakii-shi (JP)

(21) Appl. No.: 12/280,956

(22) PCT Filed: Feb. 28, 2007
(86) PCT No.: PCT/JP2007/054374

§ 371 Date: Jan. 7, 2009

Related U.S. Application Data
(60) Provisional application No. 60/778,079, filed on Feb. 28, 2006.

(51) Int. Cl. A61K 39/00; C07K 16/18; C12N 5/06

(52) U.S. Cl. 424/184.1; 530/387.7; 435/375

(57) ABSTRACT

The present invention relates to the use of cytotoxicity based on the effector function of anti-CDH3 antibodies. Specifically, the present invention provides methods and pharmaceutical compositions that comprise an anti-CDH3 antibody as an active ingredient for damaging CDH3-expressing cells using antibody effector function. Since CDH3 is strongly expressed in pancreatic, lung, colon, prostate, breast, gastric or liver cancer cells, the present invention is useful in pancreatic, lung, colon, prostate, breast, gastric or liver cancer therapies.

This application claims the benefit of U.S. Provisional Application Ser. No. 60/778,079 filed Feb. 28, 2006, the contents of which are hereby incorporated by reference in its entirety.

### FIELD OF THE INVENTION

The present invention relates to methods for damaging cells using the effector function of anti-CDH3 antibodies, or to compositions for this purpose.

### BACKGROUND OF THE INVENTION

The mortality among patients with pancreatic cancer is worse than for any other kind of malignant tumor, with a 5-year survival rate only 4% (Greenlee et al., (2001) CA. Cancer J. Clin.; 51: 15-36.). The poor prognosis of this malignancy reflects both the difficulty of early diagnosis and a generally poor response to current therapies (DiMagno et al., (1999) Gastroenterology.; 117: 1464-84; Greenlee et al., (2001) CA. Cancer J. Clin.; 51: 15-36.). In particular, no tumor marker is clinically available for detection of this disease at an early and potentially curative stage. Surgical resection is the only possible cure at present, but cases that are surgically resectable at diagnosis account for fewer than 20% of patients with this cancer (DiMagno et al., (1999) Gastroenterology.; 117: 1464-84; Klinkenbijl et al., (1999) Ann Surg.; 230:776-82; discussion 782-4). Endoscopic ultrasonography (EUS), endoscopic retrograde cholangiopancreatography (ERCP) and spiral CT are available to screen individuals at risk for familial pancreatic cancer (Brentnall et al., (1999) Ann Intern Med.; 131: 247-55), but those approaches are not practical in terms of time and cost-effectiveness to screen every asymptomatic individual. Hence, tumor markers that are sensitive and specific for pancreatic cancer must be discovered. Almost all patients at an advanced stage fail to respond to any treatment. To overcome that situation, some clinical trials have been attempting to establish therapeutic strategies on the basis of molecular technologies. Such trials have involved, for example, an MMP inhibitor, drugs designed to inhibit Ras farnesyltransferase, and antibody-based approaches (Hao and Rowinsky, (2002) Cancer Invest.; 20:387-404.; Laheru et al., (2001) Cancer J.; 7:324-37.; Rosenberg, (2000) Drugs.; 59:1071-89.). However, so far these experiments have achieved no remarkable effects on this disease.

Lung cancer is one of the most common lethal human tumors. Non-small-cell lung cancer (NSCLC) is the most common form, accounting for nearly 80% of lung tumors (American Cancer Society, Cancer Facts and Figures 2001, Am. Chem. Soc. Atlanta). The majority of NSCLCs are not diagnosed until an advanced stage, and thus the overall 10-year survival rate has stayed low at 10%, despite recent advances in multimodality therapies (Fry et al., (1999) Cancer; 86: 1867-76). Currently, chemotherapy using platinum is considered to be a fundamental therapy for NSCLCs. However, the therapeutic action of pharmaceutical agents has not progressed beyond the point of being able to prolong the survival of advanced NSCLC patients to a certain extent ((1995) Bmj.; 311: 899-909). A number of targeting therapies are being investigated, including those that use tyrosine kinase inhibitors. However, to date, promising results have been achieved only in a limited number of patients, and in some patients, therapeutic effects have accompanied severe side effects (Kris et al., (2002) Proc Am Soc Clin Oncol.; 21: 292a (A1166)).

Colorectal carcinoma is a leading cause of cancer deaths in developed countries. Specifically, more than 130,000 new cases of colorectal cancer in the United States are reported each year. Colorectal cancer represents about 15% of all cancers. Of these, approximately 5% are directly related to inherited genetic defects. In spite of recent progress in therapeutic strategies, prognosis of patients with advanced cancers remains, very poor. Although molecular studies have revealed the involvement of alterations in tumor suppressor genes and/or oncogenes in carcinogenesis, the precise mechanisms still remain to be elucidated.

Prostate cancer (PRC) is one of the most common malignancies in men and represents a significant worldwide health problem. It is the second most frequent cause of cancer death in the United States (Greenlee, R. T., et al. (2001) CA Cancer J Clin; 51: 15-36.). Incidence of PRC is increasing steadily in developed countries according to the prevalence of Western-style diet and increasing number of senior population. Increasing number of patients also die from this disease in Japan due to adoption of a Western life style (Kuroishi, T. (1995) Klinika; 25: 43-8.). Currently, the diagnosis of PRC is based on an increased level of the serum prostate specific antigen (PSA). Early diagnosis provides an opportunity for curative surgery. Patients with organ confined PRC are usually treated and approximately 70% of them are curable with radical prostatectomy (Roberts, W. W, et al. (2001) Urology; 57: 1033-7.; Roberts, S. G., et al. (2001) Mayo Clin Proc; 76: 576-81.). Most of patients with advanced or relapsed disease are treated with androgen ablation therapy because growth of PRC is initially androgen dependent. Although most of these patients initially respond to androgen ablation therapy, the disease eventually progresses to androgen-independent PRC, at which point the tumor is no longer responsive to androgen ablation therapy.

One of the most serious clinical problems of treatment for PRC is that this androgen-independent PRC is unresponsive to any other therapies, and understanding the mechanism of androgen-independent growth and establishing new therapies other than androgen ablation therapy against PRC are urgent issues for management of PRC.

Breast cancer, a genetically heterogeneous disease, is the most common malignancy in women. An estimation of approximately 800000 new cases was reported each year worldwide (Parkin D M, et al., (1999) CA Cancer J Clin; 49: 33-64). Mastectomy is the first concurrent option for the treatment of this disease. Despite surgical removal of the primary tumors, relapse at local or distant sites may occur due to undetectable micrometastasis (Saphner T, et al., (1996). J Clin Oncol; 14: 2738-46.) at the time of diagnosis. Cytotoxic agents are usually administered as adjuvant therapy after surgery aiming to kill those residual or premalignant cells.

Treatment with conventional chemotherapeutic agents is often empirical and is mostly based on histological tumor parameters, and in the absence of specific mechanistic understanding. Target-directed drugs are therefore becoming the bedrock treatment for breast cancer. Tamoxifen and aromatase inhibitors, two representatives of its kind, have been proved to have great responses used as adjuvant or chemoprevention in patients with metastasized breast cancer (Fisher B, et al. (1998) J Natl Cancer Inst; 90: 1371-88; Cuzick J, et al., (2002) Lancet; 360: 817-24). However the drawback is that only patients expressed estrogen receptors are sensitive to these drugs. A recent concerns were even raised regarding their side effects particularly lay on the possibility of causing endometrial cancer for long term tamoxifen treatment as well as deleterious effect of bone fracture in the postmenopausal women in aromatase prescribed patients (Coleman R E. (2004) Oncology.; 18 (5 Suppl 3): 16-20). Owing to the emergence of side effect and drug resistance, it is obviously necessarily to search novel molecular targets for selective smart drugs on the basis of characterized mechanisms of action.

Gastric cancer is a leading cause of cancer death in the world, particularly in the Far East, with approximately 700,000 new cases diagnosed worldwide annually. Surgery is the mainstay in terms of treatment, because chemotherapy remains unsatisfactory. Gastric cancers at an early stage can be cured by surgical resection, but prognosis of advanced gastric cancers remains very poor.

Hepatocellular carcinoma (HCC) is one of the most common cancers worldwide and its incidence is gradually increasing in Japan as well as in United States (Alkriviadis E A, et al., (1998) Br J. Surg.; 85: 1319-31). Although recent medical advances have made great progress in diagnosis, a large number of patients with HCCs are still diagnosed at advanced stages and their complete cures from the disease remain difficult. In addition, since patients with hepatic cirrhosis or chronic hepatitis have a high risk to HCCs, they may develop multiple liver tumors or new tumors even after complete removal of initial tumors. Therefore development of highly effective chemotherapeutic drugs and preventive strategies are matters of pressing concern.

Research aiming at the elucidation of carcinogenic mechanisms has revealed a number of candidate target molecules for anti-tumor agents. For example, the farnesyltransferase inhibitor (FTI) is effective in the therapy of Ras-dependent tumors in animal models (Sun J et al., (1998) Oncogene, 16:1467-73). This pharmaceutical agent was developed to inhibit growth signal pathways related to Ras, which is dependant on post-transcriptional farnesylation. Human clinical trials where anti-tumor agents were applied in combination with the anti-HER2 monoclonal antibody, trastuzumab, with the aim of antagonizing the proto-oncogene HER2/neu have succeeded in improving clinical response, and improved the overall survival rate of breast cancer patients. Tyrosine kinase inhibitor STI-571 is an inhibitor which selectively deactivates bcr-abl fusion protein. This pharmaceutical agent was developed for the therapy of chronic myeloid leukemia, where the constant activation of bcr-abl tyrosine kinase has a significant role in the transformation of white blood cells. Such pharmaceutical agents are designed to inhibit the carcinogenic activity of specific gene products (O'Dwyer M E and Druker B J, Curr Poin Oncol, 12:594-7, 2000). Thus, in cancer cells, gene products with promoted expression are usually potential targets for the development of novel anti-tumor agents.

Another strategy for cancer therapy is the use of antibodies which bind to cancer cells. The following are representative mechanisms of antibody-mediated cancer therapy:

Missile therapy: in this approach a pharmaceutical agent is bound to an antibody that binds specifically to cancer cells, and the agent then acts specifically on the cancer cells. Even agents with strong side effects can be made to act intensively on the cancer cells. In addition to pharmaceutical agents, there are also reports of approaches where precursors of pharmaceutical agents, enzymes which metabolize the precursors to an active form, and so on are bound to the antibodies.

The use of antibodies which target functional molecules: this approach inhibits the binding between growth factors and cancer cells using, for example, antibodies that bind growth factor receptors or growth factors. Some cancer cells proliferate depending on growth factors. For example, cancers dependent on epithelial growth factor (EGF) or vascular endothelial growth factor (VEGF) are known. For such cancers, inhibiting the binding between a growth factor and cancer cells can be expected to have a therapeutic effect.

Antibody cytotoxicity: antibodies that bind to some kinds of antigens can induce a cytotoxic response to cancer cells. With these types of antibodies, the antibody molecule itself induces a direct anti-tumor effect. Antibodies that display cytotoxicity to cancer cells are gaining attention as antibody agents expected to be highly effective against tumors.

### SUMMARY OF THE INVENTION

The present inventors investigated antibodies able to induce cytotoxicity, targeting genes showing increased expression in cells. The results revealed that potent cytotoxicity can be induced in CDH3-expressing cells when those cells are contacted with anti-CDH3 antibodies, thus completing the present invention.

Specifically, the present invention relates to the following pharmaceutical compositions or methods:

[1] Pharmaceutical compositions comprising an anti-CDH3 antibody as an active ingredient, wherein the anti-CDH3 antibody damages (i.e., kills the cell, is toxic to the cell, or otherwise inhibits growth or cell division), a CDH3-expressing cell using the antibody effector function.

[2] The pharmaceutical compositions are used to treat any pathological condition associated with CDH3-expressing cells. In typical embodiments, the cell is a cancer cell, such as pancreatic, lung, colorectal, prostate, breast, gastric and liver-cancer cell.

[3] The antibodies in the pharmaceutical compositions of the invention are typically monoclonal antibodies.

[4] In some embodiments, the antibody of the invention comprises an effector function such as antibody-dependent cytotoxicity, complement-dependent cytotoxicity, or both.

[5] Methods for damaging a CDH3-expressing cell will comprise the steps of:

a) contacting the CDH3-expressing cell with an anti-CDH3 antibody. As a result of the binding of the antibody the effector function of the antibody will cause damage (i.e., cytotoxicity) to the CDH3-expressing cell.

[6] Immunogenic compositions for inducing an antibody that comprises an effector function against a CDH3-expressing cell. The compositions typically comprise as an active ingredient, a CDH3 polypeptide, an immunologically active fragment thereof, or a nucleic acid molecule the expresses the polypeptides or fragments.

[7] Methods for treating disease using an antibody that comprises an effector function against a CDH3-expressing cell, wherein the method comprises administering a CDH3 polypeptide, an immunologically active fragment thereof, or a cell or a DNA that can express the polypeptides or fragments.

### BRIEF DESCRIPTION OF THE DRAWINGS

FIG. 1 is photographs depicting the result of Semiquantitative RT-PCR analysis for the CDH3 gene in cancer cells. A; for pancreatic cancer cell lines. B; for lung cancer cell lines. C; colorectal cancer cell lines. D; prostate cancer cell lines. E; breast cancer cell lines. F; gastric cancer cell lines. G; liver cancer cell lines.

FIG. 2 shows the results of an ADCC assay using Herceptin against A; KLM-1 over-expressed c-erbB-2 gene and B; PK-45P low-expressed c-erbB-2 gene.

FIG. 3 shows the results of an ADCC assay using anti-CDH3 polyclonal antibody BB039 against CDH3-over-expressing A; pancreatic cancer cell line KLM-1, B; lung cancer cell line CNI-H358, C; colorectal cancer cell line HCT-116, D; prostate cancer cell line PC-3, E and F; breast cancer cell line HCC1143 and HCC1937, G; gastric cancer cell line MKN7, H; liver cancer cell line SNU-449, and I; low-expressing pancreatic cancer cell line, PK-45P, respectively.

### DETAILED DESCRIPTION OF THE INVENTION

The present invention relates to pharmaceutical compositions for damaging CDH3-expressing cells using antibody effector function, wherein the compositions comprise an anti-CDH3 antibody as an active ingredient. The present invention also relates to use of an anti-CDH3 antibody to produce pharmaceutical compositions for damaging CDH3-expressing cells using the anti-CDH3 antibody effector function. The pharmaceutical compositions of the present invention comprise anti-CDH3 antibodies and pharmaceutically acceptable carriers.

The present inventors used cDNA microarrays for gene expression analysis of pancreatic cancer cells and normal cells collected from pancreatic cancer patients (Nakamura et al., (2004) Oncogene; 23: 2385-400). A number of genes with specifically enhanced expression in pancreatic cancer cells were subsequently identified. Of these genes with altered expression in pancreatic cancer cells, one gene, placental cadherin (P-cadherin; CDH3) gene encoding cytoplasmic membrane protein with low levels of expression in major organs was selected as a target gene for pancreatic cancer therapies. By selecting genes with low levels of expression in major organs, the danger of side effects is avoided. Among the protein encoded by the genes selected in this way, anti-CDH3 antibodies were confirmed to have effector functions against CDH3-expressing cells. In addition, a similar effect was confirmed in other cancer cell lines, such as the lung, colorectal, prostate, breast, gastric and liver-cancer cell lines that this gene over-expressed.

The findings obtained by the present inventors show that, in a forced expression system, CDH3 tagged with c-myc-His was localized in cytoplasmic membrane, which was confirmed using an immuno-fluorescence microscopy. The CDH3 gene encodes an amino acid sequence expected to comprise a signal peptide at its N-terminal. As mentioned above, this protein was observed to be chiefly localized in the cytoplasmic membrane, and thus it was thought to be a transmembrane protein. In addition, the low expression level of this gene in major organs, and its high expression in pancreatic, lung, colorectal, prostate, breast, gastric and liver-cancer cells, establishes that CDH3 is useful as a clinical marker and therapeutic target.

Preferred conditions for destroying cancer cells using effector function are, for example, the following:
Expression of large numbers of antigenic molecules on the membrane surface of cancer cells,
Uniform distribution of antigens within cancerous tissues,
Lingering of antigens bound to antibodies on the cell surface for a long time.

More specifically, for example, antigens recognized by antibodies must be expressed on the surface of the cell membrane. In addition, it is preferable that the ratio of antigen-positive cells is as high as possible in cells forming cancerous tissues. In an ideal situation, all cancer cells are antigen-positive. When antigen-positive and negative cells are mixed in cancer cell populations, the clinical therapeutic effect of the antibodies may not be expected.

Usually, when as many molecules as possible are expressed on the cell surface, potent effector functions can be expected. It is also important that antibodies bound to antigens are not taken up into cells. Some receptors are taken up into cells (endocytosis) after binding to a ligand. Equally, antibodies bound to cell surface antigens can also be taken up into the cell. This kind of phenomenon, whereby antibodies are taken up into cells, is called internalization. When internalization occurs, the antibody constant (Fc) region is taken up into the cell. However, cells or molecules essential to effector function are outside the antigen-expressing cells. Thus, internalization inhibits antibody effector function. Therefore, when expecting antibody effector function, it is important to select an antigen that causes less antibody internalization. The present inventors revealed for the first time that CDH3 is a target antigen possessing such a property.

An “isolated” or “purified” polypeptide is a polypeptide that is substantially free of cellular material such as carbohydrate, lipid, or other contaminating proteins from the cell or tissue source from which the protein is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining of the gel. In a preferred embodiment, antibodies of the present invention or fragments thereof are isolated or purified.

An “isolated” or “purified” nucleic acid molecule is one which is separated from other nucleic acid molecules which are present in the natural source of the nucleic acid molecule. An “isolated” or “purified” nucleic acid molecule, such as a cDNA molecule, can be substantially free of other cellular material or culture medium when produced by recombinant techniques, or substantially free of chemical precursors or other chemicals when chemically synthesized. In a preferred embodiment, nucleic acid molecules encoding antibodies of the present invention or fragments thereof are isolated or purified.

“Antibodies” and “immunoglobulins” are glycoproteins having the same general structural characteristics. While antibodies exhibit binding specificity to a specific antigen, immunoglobulins include both antibodies and other antibody-like molecules, for which antigen specificity has not been defined. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains (C_{H}). Each light chain has a variable domain at one end (V_{L}) and a constant domain at its other end (C_{L}); the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., (1985) J Mol Biol.; 186: 651-63; Novotny and Haber, (1985) Proc Natl Acad Sci USA.; 82: 4592-6). Antibodies used in the invention can be either native antibodies or the product of recombinant expression or other manipulations, as described below.

The term “variable domain” refers to certain portions of antibodies that differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four framework regions, largely adopting a P-sheet configuration, connected by three CDRs, which form loops connecting, and in some cases forming part of the β-sheet structure. The CDRs in each chain are held together in close proximity by the framework regions and, with the CDRs from the other chain, contribute to the formation of the antigen-binding site of antibodies (Kabat et al., (1991) Sequences of Proteins of Immunological Interest, Fifth Edition, National Institute of Health, Bethesda, Md.). The constant domains are not involved directly in binding an antibody to an antigen but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment. Pepsin treatment yields an F(ab′)₂ fragment that has two antigen-binding sites. “Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H}-1) of the heavy chain. Fab′ fragments differ from Fab fragments by the addition of a few residues at the carboxy terminus of the heavy chain C_{H}-1 domain including one or more cysteines from the antibody hinge region. Fab′-SH is the designation herein for Fab′, in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called κ (kappa) and λ (lambda), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they can be synthesized by hybridoma culture, uncontaminated by other immunoglobulins. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler and Milstein, (1975) Nature.; 256:495-7, or can be made by recombinant DNA methods (Cabilly et al., (1984) Proc Natl Acad Sci USA.; 81:3273-7).

The monoclonal antibodies herein specifically include “chimeric” antibodies or immunoglobulins, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., (1984) Proc Natl Acad Sci USA.; 81:6851-5). Most typically, chimeric antibodies or immunoglobulins comprise human and murine antibody fragments, generally human constant and mouse variable regions.

“Humanized” forms of non-human (e.g., murine) antibodies are specific chimeric immunoglobulins, immunoglobulin chains or fragments thereof which contain minimal sequence derived from non-human immunoglobulin. Such fragments also includes Fv, Fab, Fab′, F(ab′)₂, or other antigen-binding subsequences of antibodies. For the most part, humanized antibodies are human immunoglobulins (recipient antibody) in which residues from a complementarity-determining region (CDR) of the recipient are replaced by residues derived from a CDR of a non-human species (donor antibody) such as mouse, rat, or rabbit having the desired specificity, affinity, and capacity. In some instances, Fv framework residues of the human immunoglobulin may be replaced by corresponding non-human residues. In the present invention, few, two, or preferably one of framework(s) in the humanized antibody may be replaced by that of non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin. For further details see Jones et al., (1986) Nature.; 321:522-5; Riechmann et al., (1988) Nature.; 332:323-7; Presta, (1992) Curr Opin Struct Biol. 2:593-6.

Fully human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom & Winter, J. Mol. Biol. 227:381-8 (1991)), Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. A number of methods have been described to discern chemical structures for converting the naturally aggregated but chemically separated light and heavy polypeptide chains from an antibody V region into an sFv molecule which will fold into a three dimensional structure substantially similar to the structure of an antigen-binding site (U.S. Pat. Nos. 5,091,513, 5,132,405, and 4,946,778; Pluckthun in The Pharmacology of Monoclonal Antibodies, vol. 113, Rosenburg and Moore eds., Springer-Verlag, New York, pp. 269-315 (1994)). “Effector function” in the present invention refers to cytotoxicity involved with the Fc regions of antibodies. Alternatively, effector function can also be explained as a role that determines the biological activity triggered by antigen recognition of an antibody. For example, functions that drive the effect whereby the Fc regions of antibodies bound to antigens damage cells comprising those antigens, can also be referred to as antibody effector function. Herein, preferable target cells are cancer cells. Specifically, Antibody Dependent Cell-mediated Cytotoxicity (ADCC), Complement Dependent Cytotoxicity (CDC), and neutralizing activity are known as antibody effector functions. Each function is described below.

Antibody Dependent Cell-Mediated Cytotoxicity (ADCC):

Effector cell functions carried out by the Fc regions of various antibodies rely heavily on antibody class. Cells exist which comprise Fc receptors specific to the Fc region of immunoglobulin classes IgQ IgE, or IgA. The Fc region of IgQ IgE, and IgA class antibodies each binds to a specific Fc receptor, and cells that comprise a corresponding Fc receptor recognize and bind to antibodies bound to cell membranes or so on. As a result, for example, cells that have Fc receptors are activated, and function in intercellular antibody transport.

For example, an IgG class antibody is recognized by Fc receptors on T cells, NK cells, neutrophils, and macrophages. These cells bind to and are activated by the Fc region of IgG class antibodies, and express cytotoxicity against cells to which these antibodies have bound. Cells such as T cells, NM cells, neutrophils, macrophages, which acquire cytotoxicity via antibody effector function, are called effector cells. In particular, IgG class antibodies activate effector cells via Fc receptors on these cells, and then kill target cells to which the variable regions of the antibodies are bound. This is called antibody-dependent cell-mediated cytotoxicity (ADCC). ADCC may be divided based on the type of effector cell, as follows:

ADMC: IgG-dependent macrophage-mediated cytotoxicity, and

ADCC: IgG-dependent NK-cell-mediated cytotoxicity.

There is no limitation on types of effector cells in the ADCC of the present invention. In other words, the ADCC of the present invention also comprises ADMC, where macrophages are the effector cells.

Antibody ADCC is known to be an important mechanism of the anti-tumor effects, particularly in cancer therapies that use antibodies (Clynes R A, et al., (2000) Nature Med., 6: 443-6.). For example, a close relationship between the therapeutic effect of anti-CD20 antibody chimeric antibodies and ADCC has been reported (Cartron G. et al., (2002) Blood, 99: 754-8.). Thus ADCC is also particularly important among antibody effector functions in the present invention.

For example, ADCC is thought to be an important mechanism in the anti-tumor effects of Rituxan, Herceptin, and so on, for which clinical application has already begun. Rituxan and Herceptin are therapeutic agents for non-Hodgkin's lymphoma and metastatic breast cancer, respectively.

At present, the mechanism for ADCC-mediated cytotoxicity is roughly explained as follows: effector cells, which are bridged to target cells via antibodies bound to the cell surface, are thought to induce target cell apoptosis by transmitting some sort of lethal signal to the target cells. In any case, antibodies that induce cytotoxicity by effector cells are comprised in the antibodies that comprise effector function of the present invention.

To assess ADCC activity of a molecule of interest, an in vitro ADCC assay, such as that described in U.S. Pat. No. 5,500,362 or U.S. Pat. No. 5,821,337 may be performed. Alternatively, or additionally, ADCC activity of the molecule of interest may be assessed in vivo, e.g., in a animal model such as that disclosed in Clynes et al., (1998) Proc Natl Acad Sci USA.; 95:652-56.

Complement Dependent Cytotoxicity (CDC):

The Fc regions of immunoglobulins bound to antigens are known to activate complementary pathways. It has also been revealed that the activation pathway may differ depending on the class of immunoglobulin. For example, of the human antibodies, IgM and IgG activate the classical pathway. On the other hand, IgA, IgD, and IgE do not activate this pathway. Namely, the function of activating complement is limited to IgM and IgG class antibodies. Particularly, the function of lysing cells to which antibody variable regions are bound is called complement-dependent cytotoxicity (CDC).

The activated complements produce, via a number of reactions, a C5b-9 membrane attack complex (MAC) comprising cell membrane-damaging activity. MACs generated in this way are thought to damage viral particles and cell membranes, independently of effector cells. The mechanism for MAC-mediated cytotoxicity is based on the following. MACs comprise a strong binding affinity for cell membranes. MACs bound to a cell membrane open a hole in the cell membrane, making it easy for water to flow in and out of the cell. As a result, the cell membrane is destabilized, or the osmotic pressure is changed, and the cell is destroyed. Cytotoxicity due to an activated complement only extends to membrane close to the antibody which has bound the antigen. For this reason, MAC-mediated cytotoxicity is dependent on antibody specificity. ADCC and CDC can express cytotoxicity independent of each other. However, in practice, these cytotoxicities may function in composite in living bodies.

To assess CDC activity of a molecule of interest, a CDC assay, e.g., as described in Gazzano-Santoro et al., (1997) J Immunol Methods.; 202: 163-71, may be performed.

Neutralizing Activity:

Antibodies exist which have the function of depriving infectivity of pathogens and activity of toxins. Antibody-mediated neutralization can be achieved by binding of an antigenic variable region to an antigen, or can require complement mediation. For example, in some cases, anti-viral antibodies require complement mediation in order to deprive a virus of its infectivity. Fc regions are essential to the participation of complements. Thus, such antibodies comprise effector function that requires Fc for neutralizing viruses and cells.

Of these, preferable effector functions herein are either ADCC or CDC, or both. The present invention is based on the finding that anti-CDH3 antibodies bind to CDH3-expressing cells, and then express effector function.

The present invention also relates to methods for damaging CDH3-expressing cells, which comprise the following steps:

1) contacting the CDH3-expressing cells with anti-CDH3 antibodies, and

2) damaging the CDH3-expressing cells using the effector function of the antibodies which have bound to the cells.

In the methods or pharmaceutical compositions of the present invention, any CDH3-expressing cell can be damaged or killed. For example, pancreatic, lung, colorectal, prostate, breast, gastric and liver-cancer cells are preferable as the CDH3-expressing cells of the present invention. Of these, pancreatic carcinoma, non-small cell lung cancer (NSCLC), colorectal carcinoma, prostate carcinoma, breast duct carcinoma, tubular adenocarcinoma of the stomach, hepatocellular carcinoma (HCC), or cells are preferable.

Cells and antibodies can be contacted in vivo or in vitro. When targeting in vivo cancer cells as the CDH3-expressing cells, the methods of the present invention are in fact therapeutic methods or preventative methods for cancers. Specifically, the present invention provides therapeutic methods for cancers which comprise the following steps:

1) administering an antibody that binds CDH3 to a cancer patient, and

2) damaging cancer cells using the effector function of the antibody bound to those cells.

The present inventors confirmed that antibodies binding CDH3 effectively damage CDH3-expressing cells, in particular, pancreatic, lung, colon, prostate, breast, gastric or liver cancer cells using effector function. The present inventors also confirmed that CDH3 is highly expressed in pancreatic, lung, colorectal, prostate, breast, gastric and liver-cancer cells, with a high probability. In addition, CDH3 expression levels in normal tissues are low. Putting this information together, methods of pancreatic, lung, colon, prostate, breast, gastric or liver cancer therapy where anti-CDH3 antibody is administered can be effective, with little danger of side effects.

Antibodies comprising the Fc region of IgA, IgE, or IgG are preferable for expressing ADCC. Equally, the antibody Fc region of IgM or IgG is preferable for expressing CDC. However, the antibodies of the present invention are not limited so long as they comprise a desired effector function. Variants, analogs or derivatives of the Fc portion may be constructed by, for example, making various substitutions of residues or sequences.

Variant (or analog) polypeptides include insertion variants, wherein one or more amino acid residues supplement an Fc amino acid sequence. Insertions may be located at either or both termini of the protein, or may be positioned within internal regions of the Fc amino acid sequence. Insertional variants with additional residues at either or both termini can include, for example, fusion proteins and proteins including amino acid tags or labels. For example, the Fc molecule may optionally contain an N-terminal Met, especially when the molecule is expressed recombinantly in a bacterial cell such as E. coli.

In Fc deletion variants, one or more amino acid residues in an Fc polypeptide are removed. Deletions can be effected at one or both termini of the Fc polypeptide, or with removal of one or more residues within the Fc amino acid sequence. Deletion variants, therefore, include all fragments of an Fc polypeptide sequence.

In Fc substitution variants, one or more amino acid residues of an Fc polypeptide are removed and replaced with alternative residues. In one aspect, the substitutions are conservative in nature, however, the invention embraces substitutions that are non-conservative.

Preferably, the parent polypeptide Fc region is a human Fc region, e.g., a native sequence human Fc region human IgG₁ (A and non-A allotypes) or human IgG₃ Fc region. In one embodiment, the variant with improved ADCC mediates ADCC substantially more effectively than an antibody with a native sequence IgG₁ or IgG₃ Fc region and the antigen-binding region of the variant. Preferably, the variant comprises, or consists essentially of, substitutions of two or three of the residues at positions 298, 333 and 334 of the Fc region. The numbering of the residues in an immunoglobulin heavy chain is that of the EU index as in Kabat et al., (supra), expressly incorporated herein by reference. Most preferably, residues at positions 298, 333 and 334 are substituted, (e.g., with alanine residues). Moreover, in order to generate the Fc region variant with improved ADCC activity, one will generally engineer an Fc region variant with improved binding affinity for FcγRIII, which is thought to be an important FcR for mediating ADCC. For example, one may introduce an amino acid modification (e.g., an insertion, a deletion, or a substitution) into the parent Fc region at any one or more of amino acid positions 256, 290, 298, 312, 326, 330, 333, 334, 360, 378 or 430 to generate such a variant. The variant with improved binding affinity for FcγRIII may further have reduced binding affinity for FcγRII, especially reduced affinity for the inhibiting FcγRIIb receptor.

In any event, any variant amino acid insertions, deletions and/or substitutions (e.g., from 1-50 amino acids, preferably, from 1-25 amino acids, more preferably, from 1-10 amino acids) are contemplated and are within the scope of the present invention. Conservative amino acid substitutions will generally be preferred. Furthermore, alterations may be in the form of altered amino acids, such as peptidomimetics or D-amino acids.

Alternatively, in the present invention, ADCC activity may be enhanced by modifying the biochemical properties other than amino acid sequence, such as sugar-chain added to Fc region. For example, it was reported that the absence of fucose residue of IgG may enhances ADCC activity (Shinkawa et al., J. Biol. Chem., Vol. 278, No. 5, pp. 3466-3473, 2003). Therefore, antibody lacking the fucose residue of Fc region is preferable antibody of the present invention. More specifically, in order to enhance the ADCC activity, fucose residue attached to CH2 domain of Fc region may be removed. Cells other than CHO may be used as host cell for expression of an antibody lacking the fucose residue of Fc region. Fucose residue was added to antibody by alpha-1,6-fucosyl transferase (FUT8), which is highly expressed in CHO.

Therefore, human-derived antibodies belonging to these classes are preferable in the present invention. Human antibodies can be acquired using antibody-producing cells harvested from humans, or chimeric animals transplanted with human antibody genes (Ishida I, et al., (2002) Cloning and Stem Cells., 4: 91-102.).

Furthermore, antibody Fc regions can link with arbitrary variable regions. Specifically, chimeric antibodies wherein the variable regions of different animal species are bound to human constant regions are known. Alternatively, a human-human chimeric antibody can also be acquired by binding human-derived variable regions to arbitrary constant regions. In addition, CDR graft technology, where complementarity determining regions (CDRs) composing human antibody variable regions are replaced with CDRs of heterologous antibodies, is also known (“Immunoglobulin genes” (1989) Academic Press (London), pp 260-274; Roguska M A. et al., (1994) Proc Natl Acad Sci USA, 91: 969-73). By replacing CDRs, antibody binding specificity is replaced. That is, human CDH3 will be recognized by humanized antibodies in which the CDR of human CDH3-binding antibodies has been transferred. The transferred antibodies can also be called humanized antibodies. Antibodies thus-obtained and equipped with an Fc region essential to effector function can be used as the antibodies of the present invention, regardless of the origin of their variable regions. For example, antibodies comprising a human IgG Fc are preferable in the present invention, even if their variable regions comprise an amino acid sequence derived from an immunoglobulin of another class or another species.

The antibodies of the present invention may be monoclonal antibodies or polyclonal antibodies. Even when administering to humans, human polyclonal antibodies can be derived using the above-mentioned animals transferred with a human antibody gene. Alternatively, immunoglobulins which have been constructed using genetic engineering techniques, such as humanized antibodies, human-non-human chimeric antibodies, and human-human chimeric antibodies, can be used. Furthermore, methods for obtaining human monoclonal antibodies by cloning human antibody-producing cells are also known.

CDH3, or a fragment comprising its partial peptide, can be used as immunogens to obtain the antibodies of the present invention. The CDH3 of the present invention can be derived from any species, preferably from a mammal such as a human, mouse, or rat, and more preferably from a human. The human CDH3 nucleotide sequence and amino acid sequence are known. The cDNA nucleotide sequence of CDH3 (GenBank Accession No. BC041846) is described in SEQ ID NO: 1 and the amino acid sequences coded by that nucleotide sequence is described in SEQ ID NO: 2 (GenBank Accession No. AAH41846.1). One skilled in the art can routinely isolate genes comprising the provided nucleotide sequence, preparing a fragment of the sequence as required, and obtain a protein comprising the target amino acid sequence.

For example, the gene coding the CDH3 protein or its fragment can be inserted into a known expression vector, and used to transform host cells. The desired protein, or its fragment, can be collected from inside or outside host cells using arbitrary and standard methods, and can also be used as an antigen. In addition, proteins, their lysates, and chemically-synthesized proteins can be used as antigens. Furthermore, cells expressing the CDH3 protein or a fragment thereof can themselves be used as immunogens.

When using a peptide fragment as the CDH3 immunogen, it is particularly preferable to select an amino acid sequence which comprises a region predicted to be an extra-cellular domain. The existence of a signal peptide is predicted from positions 1 to 26 on the N-terminal of CDH3. (Shimoyama Y, et al., (1989) J. Cell Biol.; 109(4 Pt 1): 1787-94.) Thus, for example, a region other than the N-terminal signal peptide (26 amino acid residues) is preferred as the immunogen for obtaining the antibodies of the present invention. That is to say, antibodies that bind to CDH3 extra-cellular domains are preferred as the antibodies of the present invention.

Therefore, preferable antibodies in the present invention are antibodies equipped with an Fc essential to effector function, and a variable region that can bind to an extracellular domain. When aiming for administration to humans, it is preferable to be equipped with an IgG Fc.

Any mammal can be immunized with such an antigen. However, it is preferable to consider compatibility with parent cells used in cell fusion. Generally, rodents, lagomorphs, or primates are used.

Rodents include, for example, mice, rats, and hamsters. Lagomorphs include, for example, rabbits. Primates include, for example, catarrhine (old world) monkeys such as Macaca fascicularis, Macaca mulatta, Sacred baboons, and chimpanzees.

Methods for immunizing animals with antigens are well known in the field. Intraperitoneal or subcutaneous antigen injections are standard methods for immunizing mammals. Specifically, antigens can be diluted and suspended in an appropriate amount of phosphate buffered saline (PBS), physiological saline, or so on. As desired, antigen suspensions can be mixed with an appropriate amount of a standard adjuvant such as Freund's complete adjuvant, and administered to mammals after emulsification. Subsequently, it is preferable that antigens mixed with an appropriate amount of Freund's incomplete adjuvant are administered in multiple doses every four to 21 days. An appropriate carrier can also be used for immunization. After carrying out immunization as outlined above, standard methods can be used to examine serum for an increase in the desired antibody level.

Polyclonal antibodies against the CDH3 protein can be prepared from immunized mammals whose serum has been investigated for an increase in the desired antibodies. This can be achieved by collecting blood from these animals, or by using an arbitrary, usual method to isolate serum from their blood. Polyclonal antibodies comprise serum that comprises polyclonal antibodies, and fractions that comprise polyclonal antibodies which can be isolated from serum. IgG and IgM can be prepared from fractions that recognize CDH3 protein by using, for example, an affinity column coupled to CDH3 protein, and then further purifying this fraction using protein A or protein G columns. In the present invention, antiserum can be used as is as polyclonal antibodies. Alternatively, purified IgG, IgM, or such can also be used.

To prepare monoclonal antibodies, immunocytes are collected from mammals immunized with antigens, investigated for the increase of the desired antibody level in serum (as above), and applied in cell fusion. Immunocytes for use in cell fusion preferably come from the spleen. Other preferred parent cells for fusion with the above immunogens include, for example, mammalian myeloma cells, and more preferably, myeloma cells that have acquired properties for selection of fusion cells by pharmaceutical agents.

The above immunocytes and myeloma cells can be fused using known methods, for example the methods of Milstein et al. (Galfre, G. and Milstein, C., (1981) Methods. Enzymol, 73:3-46).

Hybridomas produced by cell fusion can be selected by culturing in a standard selective medium such as HAT medium (medium comprising hypoxanthine, aminopterin, and thymidine). Cell culture in HAT medium is usually continued for several days to several weeks, a period sufficient enough to kill all cells other than the desired hybridomas (unfused cells). Standard limiting dilutions are then carried out, and hybridoma cells that produce the desired antibodies are screened and cloned.

Non-human animals can be immunized with antigens for preparing hybridomas in the above method. In addition, human lymphocytes from cells infected with EB virus or such, can be immunized in vitro using proteins, cells expressing proteins, or suspensions of the same. The immunized lymphocytes are then fused with human-derived myeloma cells able to divide unlimitedly (U266 and so on), thus obtaining hybridomas that produce the desired human antibodies which can bind the protein (Unexamined Published Japanese Patent Application No. (JP-A) Sho 63-17688).

The obtained hybridomas are then transplanted to mice abdominal cavities, and ascites are extracted. The obtained monoclonal antibodies can be purified using, for example, ammonium sulfate precipitation, protein A or protein G columns, DEAE ion exchange chromatography, or affinity columns coupled to the proteins of the present invention. The antibodies of the present invention can be used not only in purifying and detecting the proteins of the present invention, but also as candidates for agonists and antagonists of the proteins of the present invention. These antibodies can also be applied to antibody therapies for diseases related to the proteins of the present invention. When the obtained antibodies are administered to human bodies (antibody therapy), human antibodies or humanized antibodies are preferred due to their low immunogenicity.

For example, transgenic animals comprising a repertoire of human antibody genes can be immunized with antigens selected from proteins, protein-expressing cells, or suspensions of the same. Antibody-producing cells are then recovered from the animals, fused with myeloma cells to yield hybridomas, and anti-protein human antibodies can be prepared from these hybridomas (see International Publication No. 92-03918, 94-02602, 94-25585, 96-33735, and 96-34096).

Alternatively, immunocytes such as immunized lymphocytes that produce antibodies, can be immortalized using cancer genes, and used to prepare monoclonal antibodies.

Monoclonal antibodies obtained in this way can be prepared using methods of genetic engineering (for example, see Borrebaeck, C. A. K. and Larrick, J. W., (1990) Therapeutic Monoclonal Antibodies, MacMillan Publishers, UM). For example, recombinant antibodies can be prepared by cloning DNAs that encode antibodies from immunocytes such as hybridomas or immunized lymphocytes that produce antibodies; then inserting these DNAs into appropriate vectors; and transforming these into host cells. Recombinant antibodies prepared as above can also be used in the present invention.

The antibodies can be modified by binding with a variety of molecules such as polyethylene glycols (PEGs). Antibodies modified in this way can also be used in the present invention. Modified antibodies can be obtained by chemically modifying antibodies. These kinds of modification methods are conventional to those skilled in the art. The antibodies can also be modified by other proteins. Antibodies modified by protein molecules can be produced using genetic engineering. That is, target proteins can be expressed by fusing antibody genes with genes that code for modification proteins. For example, antibody effector function may be enhanced on binding with cytokines or chemokines. In fact, the enhancement of antibody effector function for proteins fused with IL-2, GM-CSF, and such has been confirmed (Penichet M L, et al., (2001) Hum Antibodies., 10: 43-9). IL-2, IL-12, GM-CSF, TNF, eosinophil chemotactic substance (RANTES) and so on can be included in cytokines or chemokines that enhance effector function.

Alternatively, antibodies of the present invention can be obtained as chimeric antibodies which comprise a non-human antibody-derived variable region and a human antibody-derived constant region, or as humanized antibodies which comprise a non-human antibody-derived complementarity determining region (CDR), a human antibody-derived framework region (FR), and a constant region. Such antibodies can be produced using known methods.

The standard techniques of molecular biology may be used to prepare DNA sequences coding for the chimeric and CDR-grafted products. Genes encoding the CDR of an antibody of interest are prepared, for example, by using the polymerase chain reaction (PCR) to synthesize the variable region from RNA of antibody-producing cells (see, for example, Larrick et al., “Methods: a Companion to Methods in Enzymology”, vol. 2: page 106 (1991); Courtenay-Luck, “Genetic Manipulation of Monoclonal Antibodies” in Monoclonal Antibodies: Production, Engineering and Clinical Application; Ritter et al. (eds.), page 166 (Cambridge University Press, 1995), and Ward et al., “Genetic Manipulation and Expression of Antibodies” in Monoclonal Antibodies: Principles and Applications; Birch et al. (eds.), page 137 (Wiley-Liss, Inc., 1995)). DNA sequences coding for the chimeric and CDR-grafted products may be synthesised completely or in part using oligonucleotide synthesis techniques. Site-directed mutagenesis and polymerase chain reaction techniques may be used as appropriate. For example, oligonucleotide directed synthesis as described by Jones et al., (1986) Nature.; 321: 522-5 may be used. Also oligonucleotide directed mutagenesis of a pre-existing variable region as, for example, described by Verhoeyen et al., (1988) Science.; 239: 1534-6 or Riechmann et al., (supra) may be used. Also enzymatic filling in of gapped oligonucleotides using T4 DNA polymerase as, for example, described by Queen et al., (1989) Proc Natl Acad Sci USA.; 86:10029-33; PCT Publication WO 90/07861 may be used.

Any suitable host cell/vector system may be used for expression of the DNA sequences coding for the CDR-grafted heavy and light chains. Bacterial, e.g., E. coli, and other microbial systems may be used, in particular for expression of antibody fragments such as FAb and (Fab′)₂ fragments, and especially Fv fragments and single-chain antibody fragments, e.g., single-chain Fvs. Eucaryotic, e.g., mammalian, host cell expression systems may be used, in particular, for production of larger CDR-grafted antibody products, including complete antibody molecules. Suitable mammalian host cells include CHO cells and myeloma or hybridoma cell lines.

Antibodies obtained as above can be purified until uniform. For example, antibodies can be purified or separated according to general methods used for purifying and separating proteins. For example, antibodies can be separated and isolated using appropriately selected combinations of column chromatography, comprising but not limited to affinity chromatography, filtration, ultrafiltration, salt precipitation, dialysis, SDS polyacrylamide gel electrophoresis, isoelectric focusing, and so on (Antibodies: A Laboratory Manual, Harlow and David, Lane (edit.), Cold Spring Harbor Laboratory, 1988).

Protein A columns and Protein G columns can be used as affinity columns. Exemplary protein A columns in use include Hyper D, POROS, and Sepharose F. F (Pharmacia).

Exemplary chromatography (excluding affinity chromatography) include ion exchange chromatography, hydrophobic chromatography, gel filtration, reverse phase chromatography, and adsorption chromatography (“Strategies for Protein Purification and Characterization: A Laboratory Course Manual” Daniel R. Marshak et al., Cold Spring Harbor Laboratory Press, 1996). The chromatography can be performed according to the procedure of liquid phase chromatographies such as HPLC or FPLC.

For example, the antigen-binding activity of the antibodies of the present invention can be measured by using absorbance measurements, enzyme linked immunosorbent assays (ELISA), enzyme immunoassays (EIA), radioimmunoassays (RIA) and/or immunofluorescence methods. In ELISA, an antibody of the present invention is immobilized on a plate, a protein of the present invention is added to the plate, and then a sample comprising the desired antibody such as the culture supernatant of cells that produce the antibody or purified antibody is added. A secondary antibody that recognizes the primary antibody and has been tagged with an enzyme such as alkaline phosphatase is then added, and the plate is incubated. After washing, an enzyme substrate such as p-nitrophenyl phosphate is added to the plate, absorbance is measured, and the antigen-binding activity of the samples is evaluated. Protein fragments (C-terminal or N-terminal fragments, and such) can be used in the same way as proteins. The binding activity of the antibodies of the present invention can be evaluated using BIAcore (Pharmacia).

In addition, by following the methods outlined in the Examples, antibody effector function can also be evaluated. For example, target CDH3-expressing cells are incubated with effector cells in the presence of an antibody whose effector function is to be evaluated. If target cell destruction is detected, the antibody can be confirmed to comprise effector function that induces ADCC. The level of observed target cell destruction, in the absence of either antibodies or effector cells, can be compared as a control with the level of effector function. Cells which clearly express CDH3 can be used as the target cells. Specifically, a variety of cell lines confirmed to express CDH3 in the Examples can be used. These cell lines can be obtained from cell banks. In addition, monoclonal antibodies which comprise more powerful effector function can be selected.

In the present invention, anti-CDH3 antibodies can be administered to humans or other animals as pharmaceutical agents. In the present invention, animals other than humans to which the antibodies can be administered include mice, rats, guinea pigs, rabbits, chickens, cats, dogs, sheep, pigs, cows, monkeys, baboons, and chimpanzees. The antibodies can be directly administered to subjects, and in addition, can be formulated into dosage forms using known pharmaceutical formulation methods. For example, depending on requirements, they can be parenterally administered in an injectable form such as a sterile solution or suspension with water or other arbitrary pharmaceutically acceptable fluid. For example, this kind of compounds can be mixed with acceptable carriers or solvents, specifically sterile water, physiological saline, vegetable oils, emulsifiers, suspension agents, surfactants, stabilizers, flavoring agents, excipients, solvents, preservatives, binding agents and the like, into a generally accepted unit dosage essential for use as a pharmaceutical agent.

Other isotonic solutions comprising physiological saline, glucose, and adjuvants (such as D-sorbitol, D-mannose, D-mannitol, and sodium chloride) can be used as the injectable aqueous solution. They can also be used with appropriate solubilizers such as alcohols, specifically ethanols and polyalcohols (for example, propylene glycols and polyethylene glycol), and non-ionic surfactants (for example polysorbate 80™ or HCO-50).

Sesame oils or soybean oils can be used as an oleaginous solution, and benzyl benzoate or benzyl alcohols can be used with them as a solubilizer. Buffer solutions (phosphate buffers, sodium acetate buffers, or so on), analgesics (procaine hydrochloride or such), stabilizers (benzyl alcohol, phenols, or so on), and antioxidants can be used in the formulation. The prepared injections can be packaged into appropriate ampules.

In the present invention, the anti-CDH3 antibodies can be administered to patients, for example, intraarterially, intravenously, percutaneously, intranasally, transbronchially, locally, or intramuscularly. Intravascular (intravenous) administration by drip or injection is an example of a general method for systematic administration of antibodies to lung, colon, pancreatic, prostate, breast, gastric or liver cancer patients. Methods of locally concentrating antibody agents to the primary focus or metastatic focus in the lung include local injection using a bronchoscope (bronchoscopy) and local injection under CT guidance or with thoracoscopy. Methods of locally concentrating antibody agents to the primary focus or metastatic focus in the liver include local injection using a hepatic portal injection or arterial infusion. In addition, methods in which an intraarterial catheter is inserted near a vein that supplies nutrients to cancer cells to locally inject anti-cancer agents such as antibody agents, are effective as local control therapies for metastatic focuses as well as primary focuses of pancreatic, lung, colon, prostate, breast, gastric or liver cancer.

Although dosage and administration methods vary according to patient body weight and age, and administration method, these can be routinely selected by one skilled in the art. In addition, DNA encoding an antibody can be inserted into a vector for gene therapy, and the vector can be administered for therapy. Dosage and administration methods vary according to patient body weight, age, and condition, however, one skilled in the art can select these appropriately.

Anti-CDH3 antibodies can be administered to living bodies in an amount such that cytotoxicity based on effector function against CDH3-expressing cells can be confirmed. For example, although there is a certain amount of difference depending on symptoms, anti-CDH3 antibody dosage is 0.1 mg to 250 mg/kg per day. Usually, the dosage for an adult (of weight 60 kg) is 5 mg to 17.5 g/day, preferably 5 mg to 10 g/day, and more preferably 100 mg to 3 g/day. The dosage schedule is from one to ten times over a two to ten day interval, and for example, progress is observed after a three to six times administration.

Although the antibodies of the invention retain effector function, in some embodiments, cytotoxic agents can be linked to the antibodies using well known techniques. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin and the like. Cytotoxic agents also include radiochemicals made by conjugating radioisotopes to the antibodies of the invention or binding of a radionuclide to a chelating agent that has been covalently attached to the antibody. Methods for preparing such conjugates are well known in the art.

Alternatively, nucleic acids comprising sequences encoding antibodies or functional derivatives thereof, are administered to treat or prevent diseases associated with CDH3-expressing cells, such as pancreatic, lung, colon, prostate, breast, gastric, and liver cancer, by way of gene therapy. Gene therapy refers to therapy performed by the administration to a subject of an expressed or expressible nucleic acid. In this embodiment of the invention, the nucleic acids produce their encoded antibody or antibody fragment that mediates a prophylactic or therapeutic effect.

Any of the methods for gene therapy available in the art can be used according to the present invention. Exemplary methods are described below.

For general reviews of the methods of gene therapy, see Goldspiel et al., (1993) Clin. Pharm.; 12: 488-505; Wu and Wu, (1991) Biotherapy.; 3: 87-95; Tolstoshev, (1993) Ann Rev Pharmacol Toxicol.; 32: 573-96; Mulligan, (1993) Science.; 260: 926-32; Morgan and Anderson, (1993) Ann Rev Biochem.; 62:191-217; Clare Robinson, Trends Biotechnol.; 11(5):155-215. Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990).

In a preferred aspect, a composition of the invention comprises nucleic acids encoding an antibody, said nucleic acids being part of an expression vector that expresses the antibody or fragments or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, (1989) Proc Natl Acad Sci USA.; 86: 8932-5; Zijlstra et al., (1989) Nature.; 342:435-8). In specific embodiments, the expressed antibody molecule is a single chain antibody; alternatively, the nucleic acid sequences include sequences encoding both the heavy and light chains, or fragments thereof, of the antibody.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, (1987) J Biol. Chem.; 262:4429-32) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180, WO 92/22635, WO 92/20316, WO 93/14188 or WO 93/20221). Alternatively, the nucleic acid can be introduced intracellularly and incorporated within host cell DNA for expression, by homologous recombination (Koller and Smithies, (1989) Proc Natl Acad Sci USA.; 86:8932-5; Zijlstra et al., (1989) Nature.; 342:435-8).

In a specific embodiment, viral vectors that contain nucleic acid sequences encoding an antibody of the invention or fragments thereof are used. For example, a retroviral vector can be used (see Miller et al., (1993) Methods Enzymol.; 217:581-99). These retroviral vectors contain the components necessary for the correct packaging of the viral genome and integration into the host cell DNA. The nucleic acid sequences encoding the antibody to be used in gene therapy are cloned into one or more vectors, which facilitate delivery of the gene into a subject. More detail about retroviral vectors can be found in Boesen et al., (1994) Biotherapy.; 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., (1994) J Clin Invest.; 93:644-51; Kiem et al., (1994) Blood.; 83:1467-73; Salmons and Gunzberg, (1993) Hum Gene Ther.; 4:129-41; Grossman and Wilson, (1993) Curr Opin Genet Dev.; 3:110-4.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, (1993) Curr Opin Genet Dev.; 3:499-503 present a review of adenovirus-based gene therapy. Bout et al., (1994) Hum Gene Ther.; 5:3-10 demonstrates the use of adenovirus vectors to transfer genes to the respiratory epithelia of rhesus monkeys. Other instances of the use of adenoviruses in gene therapy can be found in Rosenfeld et al., (1991) Science.; 252:431-4; Rosenfeld et al., (1992) Cell.; 68:143-55; Mastrangeli et al., (1993) J Clin Invest.; 91:225-34; PCT Publication WO94/12649; Wang et al., (1995) Gene Ther.; 2:775-83. In a preferred embodiment, adenovirus vectors are used.

Adeno-associated virus (AAV) are also conveniently used in gene therapy (Walsh et al., (1993) Proc Soc Exp Biol Med.; 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, (1993) Methods Enzymol.; 217:599-618; Cotton et al., 1993, Methods Enzymol.; 217:618-44; Cline M J. Pharmacol Ther. 1985; 29(1):69-92.), and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. In a preferred embodiment, the cell used for gene therapy is autologous to the subject.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, (1992) Cell.; 71:973-85; Rheinwald, (1980) Methods Cell Biol.; 21A:229-54; Pittelkow and Scott, (1986) Mayo Clin Proc.; 61:771-7).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In addition, the present invention provides immunogenic compositions for inducing antibodies comprising effector functions against CDH3-expressing cells, where the compositions comprise as an active ingredient CDH3 or an immunologically active CDH3 fragment, or a DNA or cell which can express the same. Alternatively, the present invention relates to uses of CDH3 or an immunologically active CDH3 fragment, or a DNA or cell which can express the same in the production of immunogenic compositions for inducing antibodies comprising effector functions against CDH3-expressing cells.

The administration of anti-CDH3 antibodies damages cancer cells by the effector function of those antibodies. Thus, if anti-CDH3 antibodies can be induced in vivo, therapeutic effects equivalent to the antibody administration can be achieved. When administering immunogenic compositions comprising antigens, target antibodies can be induced in vivo. The immunogenic compositions of the present invention thus are particularly useful in vaccine therapy against CDH3-expressing cells. Thus, the immunogenic compositions of the present invention are effective as, for example, vaccine compositions for pancreatic, lung, colon, prostate, breast, gastric or liver cancer therapies.

The immunogenic compositions of the present invention can comprise CDH3 or an immunologically active CDH3 fragment, as an active ingredient. An immunologically active CDH3 fragment refers to a fragment that can induce anti-CDH3 antibodies which recognize CDH3 and comprise effector function. Below, CDH3 and the immunologically active CDH3 fragment are described as immunogenic proteins. Whether a given fragment induces target antibodies can be determined by actually immunizing an animal, and confirming the activity of the induced antibodies. Antibody induction and the confirmation of its activity can be carried out, for example, using methods described in Examples. For example, fragments comprising an amino acid sequence corresponding to CDH3 position 382 to 654 can be used as the immunogens of the present invention.

The immunogenic compositions of the present invention comprise pharmaceutically acceptable carriers as well as immunogenic proteins, the active ingredients. If necessary, the compositions can also be combined with an adjuvant. Killed tuberculosis bacteria, diphtheria toxoid, saponin and so on can be used as the adjuvant.

Alternatively, DNAs coding for the immunogenic proteins, or cells retaining those DNAs in an expressible state, can be used as the immunogenic compositions. Methods for using DNAs expressing the target antigen as immunogens, so-called DNA vaccines, are well known. DNA vaccines can be obtained by inserting a DNA encoding CDH3 or its fragment into an appropriate expression vector.

Retrovirus vectors, adenovirus vectors, adeno-associated virus vectors, Sendai virus vectors or such can be used as the vector. In addition, DNAs in which a DNA encoding an immunogenic protein is functionally connected downstream of a promoter can be directly introduced into cells as naked DNA, and then expressed. Naked DNA can be encapsulated in ribosomes or viral envelope vectors and introduced into cells.

The CDH3 polypeptides and polynucleotides of the invention can also be used for the induction of an immune response in vivo, including production of antibodies and cytotoxic T lymphocytes (CTL) specific for CDH3 expressing cells. In such methods, CTL induction by a desired peptide can be achieved by presenting the peptide to a T cell via an antigen presenting cell (APC) either in vivo or ex vivo.

For example, patient blood cells e.g., peripheral blood mononuclear cells (PBMC) are collected, transformed using a vector that can express the immunogenic proteins, and returned to the patient. Transformed bloods cells produce the immunogenic proteins inside the body of the patient, and induce the target antibodies. Alternatively, PBMCs of the patient are collected, the cells are contacted with the polypeptide ex vivo, and following the induction of APCs or CTLs, the cells may be administered to the subject. APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of tumors from other individuals.

Generally, when using a polypeptide for cellular immunotherapy, efficiency of the CTL-induction is known to be increased by combining a plurality of polypeptides having different structures and contacting them with APCs, particularly, deridritic cells. Therefore, when stimulating APCs with protein fragments, it is advantageous to use a mixture of multiple types of fragments.

The induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

When DNAs encoding the immunogenic proteins, or cells transformed with the same are used as immunogenic compositions of the present invention, they can be combined with immunogenic proteins as well as carrier proteins that enhance their immunogenic properties.

As noted above, the present invention provides methods for inducing antibodies which comprise effector function against CDH3-expressing cells, where the methods comprise the step of administering CDH3, an immunologically active CDH3 fragment, or DNA or cells that can express the same. The methods of the present invention induce antibodies that comprise effector function that damages CDH3-expressing cells such as lung, colon, pancreatic, prostate, breast, gastric or liver cancers. As a result, therapeutic effects for pancreatic, lung, colon, prostate, breast, gastric or liver cancers and so on can be obtained.

Each day, 0.1 mg to 250 mg per kilogram of the immunogenic compositions of the present invention can be administered orally or parenterally. Parenteral administration includes subcutaneous injection and intravenous injection. The administrative dose for a single adult is usually 5 mg to 17.5 g/day, preferably 5 mg to 10 g/day, and more preferably 100 mg to 3 g/day.

All prior art references cited herein are incorporated by reference in their entirety.

### EXAMPLES

Below, the present invention is further explained based on Examples.

Cell line:

Human pancreatic, lung, colon, prostate, breast, gastric or liver cancer cell lines were propagated as a monolayer in an appropriate medium with 10% or 20% fetal bovine serum. The cell lines used in the experiment are shown in Table 1.

| | | |
|---|---|---|
| cell line | Medium | Place obtained |

Pancreatic cancer Cell line

| | | |
|---|---|---|
| CAPAN1 | RPMI + 10% FBS | ATCC; HTB-79 |
| CAPAN2 | McCoy + 10% FBS | ATCC; HTB-80 |
| KLM-1 | RPMI + 10% FBS | TKG; TKG 0490 |
| MiaPaCa2 | E-MEM + 10% FBS | HSRRB; JCRB0070 |
| PK-1 | RPMI + 10% FBS | TKG; TKG 0239 |
| PK-45P | RPMI + 10% FBS | TKG; TKG 0493 |
| PK-59 | RPMI + 10% FBS | TKG; TKG 0492 |
| PK-9 | RPMI + 10% FBS | TKG; TKG 0240 |
| SUIT2 | D-MEM + 10% FBS | HSRRB; JCRB1094 |

Lung cancer Cell line

| | | |
|---|---|---|
| A549 | RPMI + 10% FBS | ATCC; CCL-185 |
| LC174 | RPMI + 10% FBS | Aichi cancer center |
| LC176 | RPMI + 10% FBS | Aichi cancer center |
| LC319 | RPMI + 10% FBS | Aichi cancer center |
| NCI-H1435 | RPMI + 10% FBS | ATCC; CRL-5875 |
| NCI-H1793 | D-MEM + 10% FBS | ATCC; CRL-5896 |
| NCI-H23 | RPMI + 10% FBS | ATCC; CRL-5800 |
| NCI-H358 | RPMI + 10% FBS | ATCC; CRL-5807 |
| NCI-H522 | RPMI + 10% FBS | ATCC; CRL-5810 |
| NCI-H596 | RPMI + 10% FBS | ATCC; HTB-178 |
| NCI-H1650 | RPMI + 10% FBS | ATCC; CRL-5883 |
| PC-14 | RPMI + 10% FBS | RIKEN Bioresource |
| | | Center |
| PC14PE6 | RPMI + 10% FBS | Tokushima University_{—} |
| PC-3 | E-MEM + 10% FBS | HSRRB; JCRB0077 |
| PC-9 | D-MEM + 10% FBS | Tokushima University_{—} |
| SK-LU-1 | E-MEM + 10% FBS + | ATCC; HTB-57 |
| | 2 mM L-glutamine | |
| SK-MES-1 | E-MEM + 10% FBS + | ATCC; HTB-58 |
| | 2 mM L-glutamine | |
| SW1573 | L15 + 10% FBS | ATCC; CRL-2170 |
| SW900 | L15 + 10% FBS | ATCC; HTB-59 |

Colorectal cancer Cell line

| | | |
|---|---|---|
| DLD-1 | RPMI + 10% FBS | ATCC; CCL-221 |
| HCT-116 | McCoy + 10% FBS | ATCC; CCL-247 |
| HCT-15 | RPMI + 20% FBS | ATCC; CCL-225 |
| HT-29 | McCoy + 10% FBS | ATCC; HTB-38 |
| LoVo | F12 + 20% FBS | ATCC; CCL-229 |
| LS174T | E-MEM + 10% FBS | ATCC; CL 188 |
| SNU-C2A | F12 + D-MEM + 10% | ATCC; CCL-250.1 |
| | FBS + 2 mM L-glutamine | |
| SNU-C4 | RPMI + 10% FBS | Korea cell-line Bank |
| SNU-C5 | RPMI + 10% FBS | Korea cell-line Bank |
| SW480 | L15 + 10% FBS | ATCC; CCL-228 |
| SW948 | L15 + 10% FBS | ATCC; CCL-237 |
| WiDr | E-MEM + 10% FBS + | ATCC; CCL-218 |
| | 2 mM L-glutamine | |

Prostate cancer Cell line

| | | |
|---|---|---|
| DU145 | E-MEM + 10% FBS + | ATCC; HTB-81 |
| | 2 mM L-glutamine | |
| LNCaP | RPMI + 10% FBS + 2 mM | ATCC; CRL-1740 |
| | L-glutamine | |
| PC-3 | E-MEM + 10% FBS | ATCC; CRL-1435 |

Breast cancer Cell line

| | | |
|---|---|---|
| BT-20 | E-MEM + 10% FBS | ATCC; HTB-19 |
| BT-474 | D-MEM + 10% FBS | ATCC; HTB-20 |
| BT-549 | RPMI + 10% FBS | ATCC; HTB-122 |
| MDA-MB-157 | L15 + 10% FBS | ATCC; HTB-24 |
| MDA-MB-231 | L15 + 10% FBS | ATCC; HTB-26 |
| MDA-MB-453 | McCoy + 10% FBS | ATCC; HTB-131 |
| MDA-MB-435S | L15 + 10% FBS | ATCC; HTB-129 |
| HCC-1143 | RPMI + 10% FBS | ATCC; CRL-2321 |
| HCC-1395 | RPMI + 10% FBS + 2 mM | ATCC; CRL-2324 |
| | L-glutamine | |
| HCC-1500 | RPMI + 10% FBS + 2 mM | ATCC; CRL-2329 |
| | L-glutamine | |
| HCC-1937 | RPMI + 10% FBS + 2 mM | ATCC; CRL-2336 |
| | L-glutamine | |
| SK-BR-3 | RPMI + 10% FBS | ATCC; HTB-30 |
| MCF-7 | E-MEM + 10% FBS | ATCC; HTB-22 |
| T47D | RPMI + 10% FBS + 2 mM | ATCC; HTB-133 |
| | L-glutamine | |
| ZR-75-1 | E-MEM + 10% FBS | ATCC; CRL-1500 |

Gastric cancer Cell line

| | | |
|---|---|---|
| MKN1 | RPMI + 10% FBS | HSRRB; JCRB0252 |
| MKN28 | RPMI + 10% FBS | HSRRB; JCRB0253 |
| MKN45 | RPMI + 10% FBS | HSRRB; JCRB0254 |
| MKN7 | RPMI + 10% FBS | HSRRB; JCRB1025 |
| MKN74 | RPMI + 10% FBS | HSRRB; JCRB0255 |
| St4 | RPMI + 10% FBS | JFCR; |
| TMK-1 | D-MEM + 10% FBS | Hiroshima Univ. |
| | | Sch. Med |

Liver cancer Cell line

| | | |
|---|---|---|
| Alexander | D-MEM + 10% FBS | HSRRB; IFO50069 |
| HepG2 | D-MEM + 10% FBS | HSRRB; JCRB1054 |
| HUH-7 | D-MEM + 10% FBS | HSRRB; JCRB0403 |
| SNU-398 | RPMI + 10% FBS (heat | ATCC; CRL-2233 |
| | inactivated) | |
| SNU-423 | RPMI + 10% FBS | ATCC; CRL-2238 |
| SNU-449 | RPMI + 10% FBS | ATCC; CRL-2234 |
| SNU-475 | RPMI + 10% FBS | ATCC; CRL-2236 |

E-MEM; Eagle's Minimal Essential medium
F-12; F-12 Nutrient Mixture (HAM)
L-15; Leibovitz's L-15 medium
McCoy; McCoy's 5A medium Modified
RPMI; RPMI 1640 medium
ATCC; American Type Culture Collection
HSRRB; Health Science Research Resources Bank
JFCR; Japanese foundation for cancer research
TKG; Institute of Development, Aging and Cancer. Tohoku University

Furthermore, the following cell lines were used in ADCC assays using anti-CDH3 antibody:

Pancreatic cancer cell line KLM-1.

Lung cancer cell line CNI-H358.

Colorectal cancer cell line HCT-116.

Prostate cancer cell line PC-3.

Breast cancer cell lines HCC1143 and HCC1937.

Gastric cancer cell line MKN7.

Liver cancer cell line SNU-449.

### Construction of Antibodies

According to standard protocols, individual protein specific antibodies were produced in Medical Biological Laboratories MBL; Nagoya, Japan) using His-tagged fusion proteins expressed in bacteria as immunogens. These fusion proteins comprised a protein portion that corresponded to one part of the protein (residues 382 to 654).

### Semiquantitative RT-PCR for CDH3:

Total RNA was extracted from the cell lines using the Rneasy® Kit (QIAGEN). In addition, mRNA was purified from total RNA by Oligo (dT)-cellulose column (Amersham Biosciences) and synthesized to first-strand cDNA by reverse transcription (RT) using the SuperScript First-Strand Synthesis System (Invitrogen). It was prepared appropriate dilutions of each first-stranded cDNA for subsequent PCR amplification by monitoring GAPDH as a quantitative control. The primer sequences the present inventors used were 5′-CTGAAGGCGGCTAACACAGAC-3′ (SEQ.ID.NO.3) and 5′-TACACGATTGTCCTCACCCTTC-3′ (SEQ.ID.NO.4) for CDH3, 5′-GTATTTGATGGTGACCTGGGAAT-3′ (SEQ.ID.NO.5) and 5′-CCCCTGGGTCTTTATTTCATCT-3′ (SEQ.ID.NO.6) for c-erbB2, 5′-GTCAGTGGTGGACCTGACCT-3′ (SEQ.ID.NO.7) and 5′-GGTTGAGCACAGGGTACTTTATT-3′ (SEQ.ID.NO.8) for GAPDH, 5′-GAGGTGATAGCATTGCTTTCG-3′ (SEQ.ID.NO.9) and 5′-CAAGTCAGTGTACAGGTAAGC-3′ (SEQ.ID.NO.10) for β-actin. All PCR reactions involved initial denaturation at 94° C. for 2 min and consisted of 94° C. for 30 s, 58° C. for 30 s, and 72° C. for 1 min by 21 cycles for GAPDH, 32 cycles for c-erbB2 (annealing temp. were lowered gradually from 62° C. to 58° C.), 20 cycles for β-actin (annealing temp. were lowered gradually from 62° C. to 57° C.) or 28 cycles for CDH3 (annealing temp. were lowered gradually from 62° C. to 56° C.) on a GeneAmp PCR system 9700 (PE Applied Biosystems).

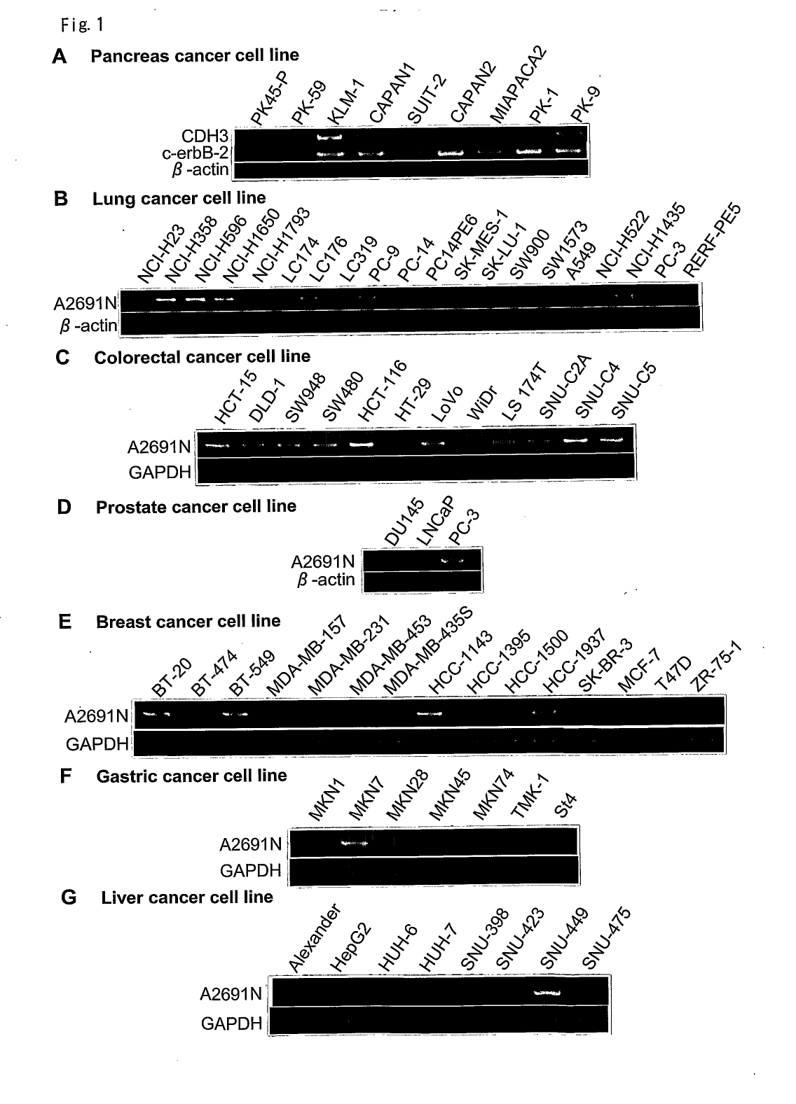

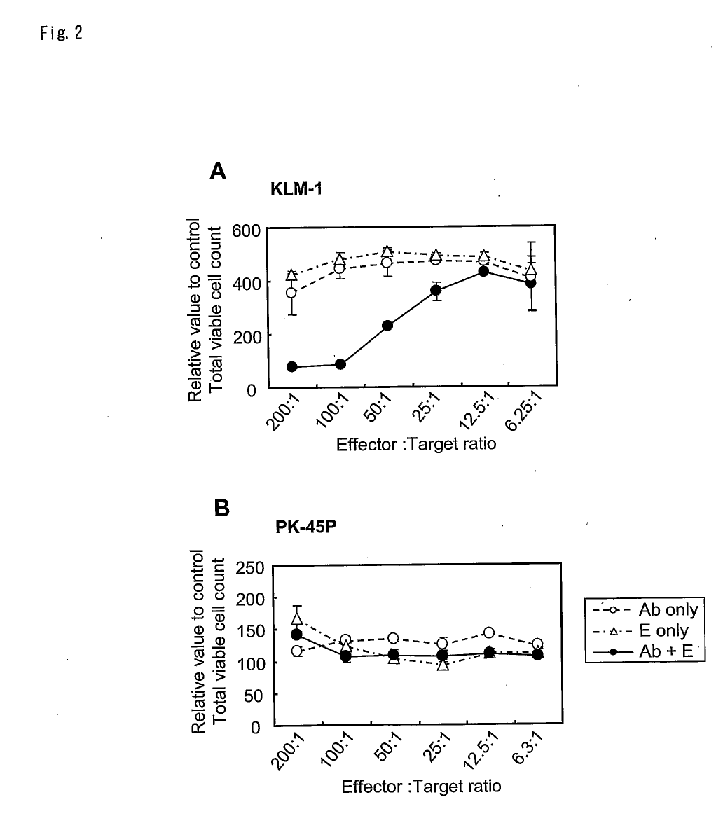

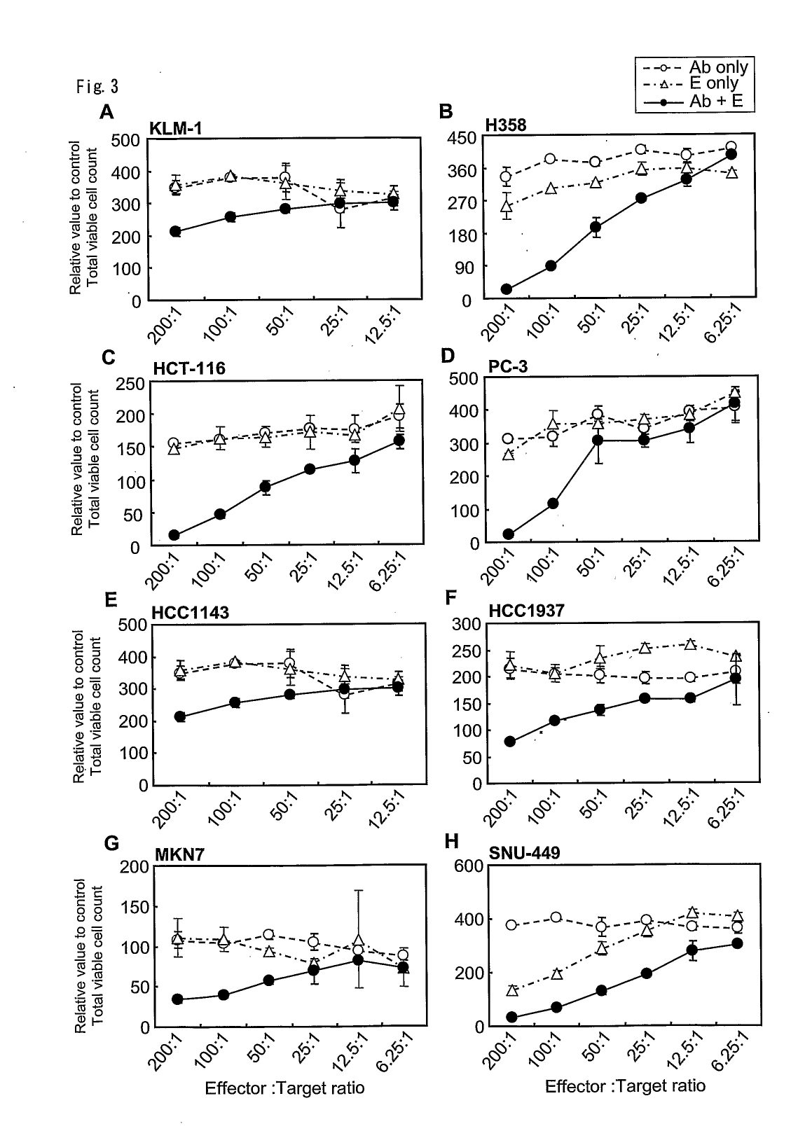

The over-expression of CDH3 was found in pancreatic cancer cell line KLM-1 (FIG. 1A). In addition, to elucidate the efficacy of anti-CDH3 polyclonal antibody (BB039) on various cancers, the expression of CDH3 was confirmed. The over-expression of CDH3 was decided in lung cancer cell line CNI-H1358, colorectal cancer cell line HCT-116, prostate cancer cell line PC-3, breast cancer cell line HCC-1143 and HCC-1937, gastric cancer cell line MKN7, liver cancer cell line SNU-449 (FIG. 1B-G).

### Flow Cytometric Analysis

The cancer cells (5×10⁶) were incubated with purified polyclonal antibodies (pAb) or rabbit IgG (control) at 4° C. for 30 min. The cells were washed in phosphophate-buffered saline (PBS) and then incubated in FITC-labeled Alexa Flour 488 (Invitrogen) at 4° C. for 30 min. The cells were washed in PBS and analyzed on a flow cytometer (FACSCalibur®, Becton Dickinson) and analyzed by BD CellQuest™ Pro software (Becton Dickinson.). Mean fluorescence intensity (MFI) was defined as ratios of flow cytometric intensity (Intensity by each protein specific antibody/Intensity by rabbit IgG).

Using CDH3 over-expressing cells, the binding ratios of anti-CDH3 antibodies on the cell surface were investigated. As a result, a higher proportion of anti-CDH3 polyclonal antibodies (BB039) bound to KLM-1, CNI-H358, HCT-116, PC-3, HCC1143 HCC1937, MKN7, SNU-449, and PK-45P cells (MFI: 124.09, 145.96, 78:44, 56.77, 151.2, 67.32, 102.7, 75.67 and 8.51, respectively) than did rabbit IgG (the control)

### ADCC Assay

After the target cells were exposed with 0.8 μM of calcein acetoxymethyl ester (Calcein-AM, DOJINDO) for 30 minutes at 37° C. Calcein-AM becomes fluorescent after the cleavage of calcein-AM by cellular esterases that produce a fluorescent derivate calcein. Target cancer cells were washed twice with AIM-V medium (Life Technologies, Inc.) before adding to the assay and then seeded hi 96-well U-bottomed plates (4×10³ cells/well). Human peripheral blood mononuclear cells (PBMCs) were obtained from healthy volunteer and separated by Ficoll-Paque (Amersham Biosciences) density gradient centrifugation and used as the effector cells. Target cancer cells and effector cells at various E: T ratios were co-incubated in 200 μl of AIM-V medium in a 96-well U-bottomed plate in triplicate for 6 hours at 37° C. with anti-CDH3 antibody BB039 (1 μg/well) or control antibody, Herceptin (10 μg/well; Roche). The ADCC effects of anti-CDH3 polyclonal antibody (BB039) for these cells were evaluated based on the fluorescent images of viable cells were rapidly acquired using the IN Cell Analyzer 1000 (Amersham Bioscience). These images were numerically converted as viable cell count (cell area for target cells) by counting the fluorescent object or vesicle using Developer tool ver. 5.21 software (Amersham Bioscience).

Control assays included the incubation of target cells with only anti-CDH3 antibody BB039 or effector cells. Herceptin was used as a control in some experiments.

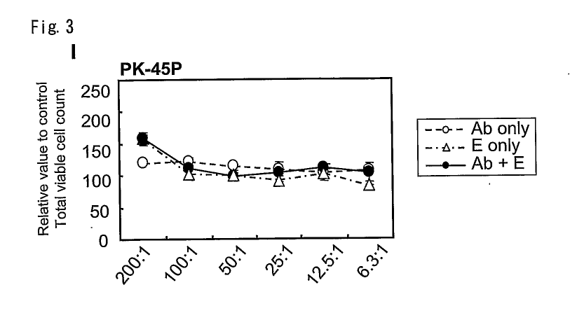

Direct cell damage of cells by BB039 anti-CDH3 polyclonal antibody itself was not observed. However, BB039 anti-CDH3 polyclonal antibody induced ADCC in KLM-1 NCI-H358, HCT-116, PC-3, HCC1143, HCC1937, MKN7 and SNU-449 cells that over-expressed CDH3 (FIG. 3A-H), while no effect against PK-45P cells with CDH3 low-expression (FIG. 31).

### INDUSTRIAL APPLICABILITY

The present invention is based, at least in part, on the discovery that CDH3-expressing cells can be damaged by antibody cytotoxicity. CDH3 was identified by the present inventors as a gene strongly expressed in pancreatic, lung, colon, prostate, breast, gastric or liver cancers. Thus, treatment of disease associated with CDH3-expressing cells, for example, pancreatic, lung, colon, prostate, breast, gastric or liver cancer is conveniently carried out using antibodies that bind to CDH3. Results actually confirmed by the present inventors show cytotoxicity due to the effect of ADCC in pancreatic, lung, colon, prostate, breast, gastric or liver cancer cell lines, in the presence of anti-CDH3 antibody.

1. A pharmaceutical composition for damaging a CDH3-expressing cell, the composition comprising an anti-CDH3 antibody as an active ingredient, wherein the antibody comprises antibody effector function.

2. The pharmaceutical composition of claim 1, wherein the CDH3-expressing cell is a pancreatic, lung, colon, prostate, breast, gastric or liver cancer cell.

3. The pharmaceutical composition of claim 1, wherein the anti-CDH3 antibody is a monoclonal antibody.

4. The pharmaceutical composition of claim 1, wherein the antibody effector function is either antibody-dependent cytotoxicity or complement-dependent cytotoxicity, or both.

5. A method for damaging a CDH3-expressing cell, comprising the steps of:
a) contacting the CDH3-expressing cell with an anti-CDH3 antibody, and
b) damaging the CDH3-expressing cell with the effector function of the antibody that has bound to the cell.

6. An immunogenic composition for inducing an antibody that comprises an effector function against a CDH3-expressing cell, wherein the composition comprises, as an active ingredient, CDH3, an immunologically active fragment thereof, or a DNA that can express CDH3 or the immunologically active fragment.

7. A method for inducing an antibody that comprises an effector function against a CDH3-expressing cell, wherein the method comprises administering CDH3, an immunologically active fragment thereof, or a cell or a DNA that can express CDH3 or the immunologically active fragment.


SEQUENCE LISTING (10 sequences)

SEQ ID NO: 1 - DNA, 3686 bp, Homo sapiens
  CDS (545)..(3034)
ggctagcgcgggaggtggagaaagaggcttgggcggccccgctgtagccgcgtgtgggaggacgcacgggcctgcttcaaagctttgggataacagcgcctccgggggataatgaatgcggagcctccgttttcagtcgacttcagatgtgtctccacttttttccgctgtagccgcaaggcaaggaaacatttctcttcccgtactgaggaggctgaggagtgcactgggtgttcttttctcctctaacccagaactgcgagacagaggctgagtccctgtaaagaacagctccagaaaagccaggagagcgcaggagggcatccgggaggccaggaggggttcgctggggcctcaaccgcacccacatcggtcccacctgcgagggggcgggacctcgtggcgctggaccaatcagcacccacctgcgctcacctggcctcctcccgctggctcccgggggctgcggtgctcaaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatagtgaccgaccagaatgaccacaagcccaagtttacccaggacaccttccgagggagtgtcttagagggagtcctaccaggtacttctgtgatgcagatgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccaaaaaaaaaaaaaaaa

SEQ ID NO: 2 - protein, 829 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQMTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 3 - DNA, 21 bp, artificial
  An artificially synthesized primer sequence for RT-PCR.
ctgaaggcggctaacacagac

SEQ ID NO: 4 - DNA, 22 bp, artificial
  An artificially synthesized primer sequence for RT-PCR.
tacacgattgtcctcacccttc

SEQ ID NO: 5 - DNA, 23 bp, artificial
  An artificially synthesized primer sequence for RT-PCR.
gtatttgatggtgacctgggaat

SEQ ID NO: 6 - DNA, 22 bp, artificial
  An artificially synthesized primer sequence for RT-PCR.
cccctgggtctttatttcatct

SEQ ID NO: 7 - DNA, 20 bp, artificial
  An artificially synthesized primer sequence for RT-PCR.
gtcagtggtggacctgacct

SEQ ID NO: 8 - DNA, 23 bp, artificial
  An artificially synthesized primer sequence for RT-PCR.
ggttgagcacagggtactttatt

SEQ ID NO: 9 - DNA, 21 bp, artificial
  An artificially synthesized primer sequence for RT-PCR.
gaggtgatagcattgctttcg

SEQ ID NO: 10 - DNA, 21 bp, artificial
  An artificially synthesized primer sequence for RT-PCR.
caagtcagtgtacaggtaagc